(19) United States
(12) Patent Application Publication
(10) Pub. No.: US 2026/0248605 A1
(43) Pub. Date: Aug. 27, 2026

(54) Soft Anchor Constructs And Methods Of Tissue Repairs

(71) Applicant: Arthrex, Inc., Naples, FL (US)

(72) Inventors: Justin M. BOYLE, Naples, FL (US); Timothy J. MCINTYRE, Naples, FL (US); Jacob A. JOLLY, Naples, FL (US); Nirav PATEL, Naples, FL (US); Michael N. SCHMIDT, Naples, FL (US)

(73) Assignee: Arthrex, Inc., Naples, FL (US)

(21) Appl. No.: 19/545,128

(22) Filed: Feb. 20, 2026

Related U.S. Application Data
(60) Provisional application No. 63/761,194, filed on Feb. 21, 2025.

(51) Int. Cl. A61F 2/08

(52) U.S. Cl. CPC A61F 2/0811; A61F 2002/0817; A61F 2002/0852; A61F 2210/0057; A61F 2220/0075

(57) ABSTRACT

Surgical anchor constructs, fixation devices, and methods for tissue repairs are disclosed. A soft suture anchor has a cannulated sheath (that is fixed in bone), at least one repair suture attached to the sheath, and at least one hybrid shuttling link attached to the sheath. The hybrid shuttling link is configured to act as both a shuttle/pull device and a repair suture. A free end of the repair suture is passed through the lumen and the body of the sheath to form at least one splice and a an adjustable, flexible, tensionable, knotless loop around soft tissue to be attached to bone. The hybrid shuttling link may form a knotted loop around soft tissue to be attached to bone.

### CROSS-REFERENCE TO RELATED APPLICATIONS

The application claims priority to U.S. Provisional Patent Application 63/761,194 filed Feb. 21, 2025, the disclosure of which is hereby incorporated be reference in its entirety herein.

### BACKGROUND

The disclosure relates to the field of surgery and, more specifically, to surgical suture constructs and methods of making, and tissue repairs for reconstructive surgeries.

### SUMMARY

Reconstruction systems, assemblies and methods for fixation of tissue are disclosed.

A surgical construct may create a knotless, self-locking, reinforced repair. A surgical construct may be made completely of suture to achieve fixation in bone without a separate anchoring body. A surgical construct may be employed in knotless fixation of first tissue to second tissue, for example, fixation of soft tissue to bone. A surgical construct may be a tensionable construct that includes a soft anchor in the form of an all-suture anchor.

In some implementations, a soft anchor may include a soft anchor sheath with one or more repair sutures and at least one shuttling link including a closed loop spaced apart from tails of the shuttling link. In some implementations, the shuttling link may include a suture with a closed loop located down the tail of the suture. In some implementations, a shuttling link may include a plurality of closed loops. In some implementations, a shuttling link may include a suture passer such as a needle. In some implementations, a shuttling link may not be discarded after conversion of the repair suture and may become part of the repair construct.

Methods of tissue repairs are also disclosed. In some implementations, knotless surgical constructs provide tissue fixation by providing an all-suture repair which is conducted with fewer steps, increased suture management, and increased reproducibility.

### DESCRIPTION OF THE DRAWINGS

FIG. 1 illustrates a surgical construct.

FIG. 2 illustrates another surgical construct.

FIG. 3 illustrates another surgical construct.

FIG. 4 illustrates another surgical construct.

FIG. 5 illustrates a schematic view of the braid shuttling suture of the surgical construct of FIG. 3.

FIGS. 6-12 illustrate an exemplary repair with surgical constructs.

### DETAILED DESCRIPTION

Tensionable, self-locking surgical constructs can create knotless, reinforced repairs.

A soft tissue repair system may include one or more tensionable constructs. In some implementations, a tensionable construct may include an all-suture anchor.

In some implementations, a tensionable construct may include a soft suture anchor with (i) one or more repair sutures (flexible strands) attached to an anchor sheath; and (ii) one or more hybrid shuttling links attached to the anchor sheath. In some implementations, a hybrid shuttling link may be employed (i) to shuttle one or more repair sutures and (ii) to aid in a surgical repair and become part of the repair construct. In some implementations, a hybrid shuttling link may include a closed loop (eyelet) provided along the shuttling link and spaced apart from most distal and proximal ends of the shuttling link, down the tails of the shuttling link. In some implementations, a hybrid shuttling link may include a plurality of closed loops (eyelets) provided along the shuttling link and spaced apart from most distal and proximal ends of the shuttling link. In some implementations, a hybrid shuttling link may include one or more needles provided at one or more ends of the shuttling link.

Methods of knotless tissue repairs are also disclosed. In some implementations, a surgical construct provides knotless first tissue to second tissue fixation, for example, fixation of soft tissue (ligament, tendon, graft, etc.) to bone without multiple knot formation and in a simple and fast manner. Methods of tissue repairs with soft anchor constructs are also disclosed. An exemplary method includes inter alia the steps of: (i) passing a free end of a flexible strand secured to a body of a cannulated soft anchor sheath through an eyelet of a hybrid shuttling link attached to the cannulated soft anchor sheath; (ii) passing the free end back through the body of the cannulated soft anchor sheath to form a splice and at least one adjustable, flexible, tensionable loop around soft tissue and through the sheath; and (iii) employing the hybrid shuttling link for one or more tissue repair applications.

The one or more tissue repair applications can include soft tissue repairs; orthopedic surgical repairs such as rotator cuff repairs, Achilles tendon repairs, patellar tendon repairs, ACL/PCL reconstructions, hip and shoulder reconstructions, and hand, wrist, foot, and ankle repairs, among many others. The one or more tissue repairs can include a knotless repair. The one or more tissue repairs can include a knotted repair. The one or more tissue repairs can include knotless and knotted repairs.

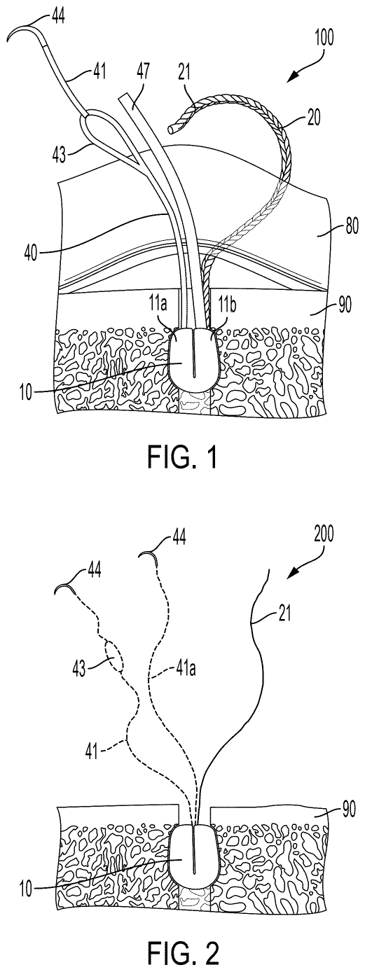

Referring now to the drawings, where like elements are designated by like reference numerals, FIGS. 1-5 illustrate exemplary fixation devices 100, 200, 300, 400. FIGS. 6-12 illustrate steps of exemplary soft tissue repair 101 with surgical systems 100, 200, 300, 400.

Fixation devices 100, 200, 300, 400 may be soft anchors. Fixation devices 100, 200, 300, 400 may be soft suture anchor constructs. Fixation devices 100, 200, 300, 400 may be surgical constructs. Fixation devices 100, 200, 300, 400 may be surgical systems. Fixation devices 100, 200, 300, 400 may be tensionable, self-locking, surgical constructs.

As detailed below, surgical constructs 100, 200, 300, 400 are formed of implantable devices made completely of suture or sutures that achieve fixation in bone without a separate anchoring body. The design enables for fixation by deployment into bone with one or more suture tails remaining outside the bone for device attachment, tensioning and/or alternative usage.

FIG. 1 illustrates a schematic view of surgical construct 100 that includes (i) a soft anchor 50 with cannulated sheath 10 and flexible strand 20 and (ii) a shuttling link 40. Cannulated sheath 10 can be in the form of a tubular sleeve or tubular member with a body made of a flexible material, such as a braided, woven, or knitted structure made of yarns, fibers, filaments, sutures or similar materials, or combinations of these materials. In some implementations, sheath 10 may be constructed of ultra-high molecular weight polyethylene (UHMWPE). In some implementations, sheath 10 may be constructed of UHMWPE and polyester. In some implementations, sheath 10 may be constructed of UHMWPE with an elastic component. In some implementations, sheath 10 may be constructed of polyester suture material with an elastic component. The elastic component may be elastane. The elastic component may be incorporated into the sheath via braiding, weaving, and/or knitting. In some implementations, an elastic sheath may include a combination of elastic, polyester, and UHMWPE all braided in a tubular jacket. An elastic sheath may consist essentially of elastane, polyester, and UHMWPE. The elastic component provides elasticity while the other components provide strength and limit the elongation of the suture.

Sheath 10 can also be any tubular or non-tubular structure having a lumen (not shown) along its length. The lumen can have a constant diameter. The lumen can have a varying diameter along its length.

Soft anchor 50 may include at least one flexible strand 20 passing through cannulated sheath 10. Sheath 10 includes a tubular body that extends between opposing ends 11a, 11b. The opposing ends 11a, 11b can be open or closed ends. The tubular body establishes the bore or lumen that extends between the opposing ends 11a, 11b. Flexible strand 20 (flexible coupler 20; suture 20) may be passed through at least a portion of bore of the sheath 10 and through the body of the sheath 10 with free end 21 exiting one of the ends 11a, 11b, or at a location close to the one of the free ends 11a, 11b. Flexible strand 20 located within the body of the sheath 10 may be fixed relative to the sheath (or affixed to the sheath 10), for example, by being stitched to the sheath 10.

Soft anchor 50 also includes a shuttling link 40. Shuttling link 40 may be a shuttle/pull device. Shuttling link 40 may be a hybrid shuttling repair suture. As detailed below, shuttling link 40 is a hybrid shuttling link that allows the surgeon to add an additional point of fixation to increase the strength of the repair construct. The hybrid link 40 functions as both a shuttling device as well as a repair suture. The hybrid link 40 allows for more advanced stitching configurations where tissue quality may be compromised. The hybrid link 40 may also function as an InternalBrace™ suture that can span to another suture anchor.

Shuttling link 40 may include a shuttle/pull device, a suture passing instrument, a suture passer, a shuttle link, a FiberLink™, a nitinol loop. Shuttling link 40 includes an eyelet/loop 43 for passing flexible coupler 20. If more than one shuttling links 40 are provided, the shuttling links can extend through the sheath 10 in similar or different directions and/or orientations and/or locations. Details of an exemplary soft suture anchor with a soft anchor sleeve (sheath or tubular member) and flexible shuttling strands are set forth, for example, in U.S. application Ser. No. 15/998,516 entitled “Methods of Tissue Repairs” filed on Aug. 16, 2018 (issued as U.S. Pat. No. 10,849,734 on Dec. 1, 2020), the disclosure of which is incorporated by reference in its entirety herein. The flexible coupler and the one or more shuttling links can extend through the sleeve in similar or different directions and/or orientations and/or locations.

Eyelet/loop 43 of shuttling link 40 is spaced apart from ends/tail 41 of the shuttling link 40. In some implementations, eyelet 43 is moved down the tail 41 of the shuttling link 40. In some implementations, at least one needle 44 is attached to tail 41 to aid in additional repairs.

Flexible coupler 20 of FIG. 1 is provided with a single terminal end 21. The terminal end 21 is a flexible end (free end 21) that may be provided integral with sheath 10. Shuttling link 40 is passed through the sheath 10. Shuttling link 40 may be attached to the sheath 10 and/or flexible coupler 20 by being spliced, for example, to the flexible coupler. Shuttling link 40 may slide freely within the sheath 10.

In some implementations, the flexible coupler 20 may be passed through at least a portion of the body of the sheath 10, for example, through a full length of the sheath, or may enter and/or exit the body of the sheath at a location other than most distal end and most proximal end of sheath 10. The sheath 10 may have any desired length and diameter and may have one or more tails or no tail. In some implementations, flexible coupler may pass through less than a full length of the sheath and may be stitched or glued within the sheath 10, with only a free end 21 outside the sheath 10.

In some implementations, soft suture anchor 50 may be a knotless hybrid FiberTak® soft anchor that may be single-loaded or multiple-loaded. As detailed below, construct 100 may be a hybrid surgical knotted and knotless construct in that it includes a knotless repair suture 20, 21 as well as a knotted repair suture 41.

Reference is now made to FIG. 2 which illustrates surgical construct 200. Construct 200 is about similar to construct 100 detailed above in that shuttling link 40 also includes a loop 43 that is moved further into the mid body portion of shuttling link 40. However, construct 200 includes an additional shuttling link 41a (tail 41a), wherein each link 41, 41a is provided with a swagged repair needle 44.

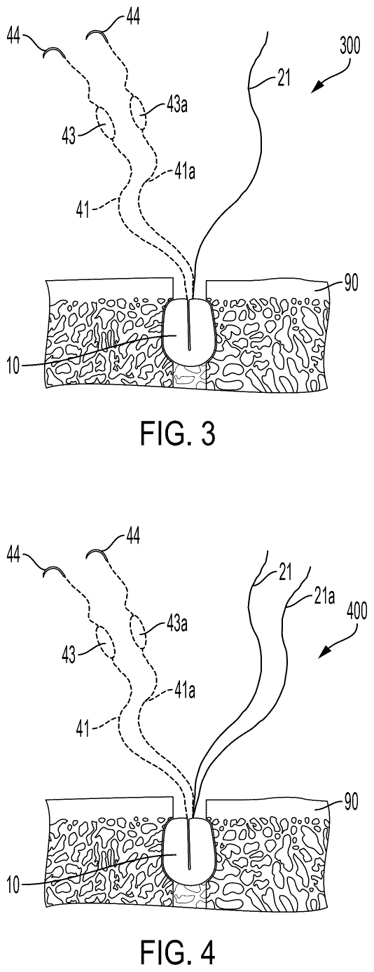

Construct 300 of FIG. 3 is about similar to construct 200 detailed above in that it includes two tails 41, 41a with tail 41 of shuttling link 40 also including a loop 43 that is moved further into the mid body portion of shuttling link 40. However, construct 300 includes a second loop 43a provided on shuttling link 41a to facilitate the ability to use the link in either direction for multiple passes through tissue or multiple repair sutures. Each link 41, 41a is provided with a swagged repair needle 44.

Construct 400 of FIG. 4 is about similar to construct 300 detailed above in that construct 400 also includes two loops 43, 43a that are moved further into the mid body portion of shuttling link 40. However, construct 400 includes a second repair suture 21a to make it a double-loaded construct. Each repair limb 21, 21a can be passed individually through the bidirectional double link 41, 41a. Each link 41, 41a is provided with a swagged repair needle 44. Repair sutures 21, 21a can be two separate and distinct sutures. Alternatively, repair sutures 21, 21a can be part of a single, bifurcated suture.

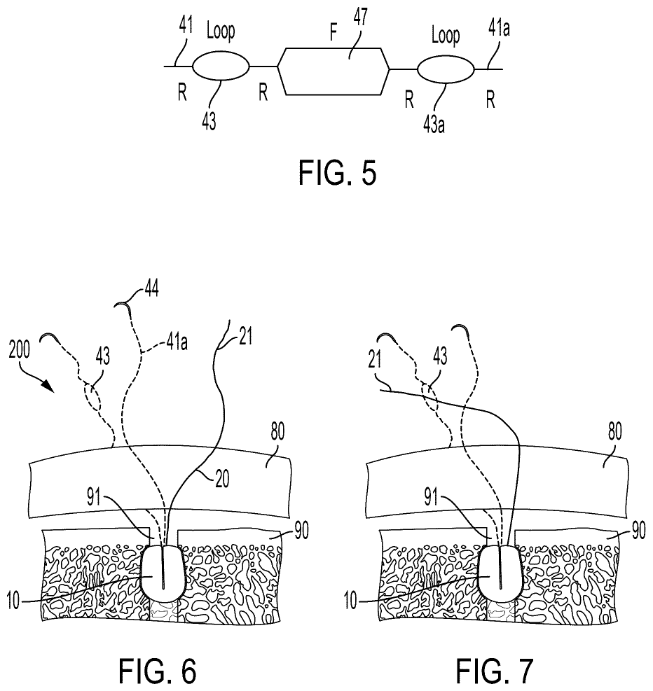

FIG. 5 illustrates a schematic view of the bidirectional double link 41, 41a of construct 400. Loop 43, 43a can be located within link 41, 41a of round suture and connected by a portion of flat suture or flat tape 47. Flat suture/tape 47 may reside fully within sheath 10. In some implementations, flat suture tape 47 may reside partially outside the sheath 10 or fully outside the sheath 10. FIG. 1 shows, for example, a portion of flat suture tape 47 exiting sheath 10.

Although the embodiments and implementations detailed above have been described with reference to a soft anchor provided with one or two repair sutures 21, 21a and one or more shuttling links 41, 41a with loops 43, 43a, it must be understood that the disclosure is not limited to these exemplary-only implementations and contemplates soft anchors provided with any number of repair sutures and shuttling links, depending on the specifics and characteristics of the surgical repair and the surgeon's preferences.

Reference is now made to FIGS. 6-12 which illustrate subsequent steps of exemplary soft tissue repair 101 that allows tissue 80 to be attached to tissue 90 with exemplary surgical construct 200 of FIG. 2. The all-suture anchor repair 101 is a tensionable repair that facilitates smaller drill tunnel holes, fewer surgical steps, and increased suture management during the repair, without the need to discard expensive surgical material forming shuttling link 40.

FIG. 6: Surgical construct 200 is inserted into bone hole 91 of bone 90 for attachment of soft tissue 80. Sheath 10 is located fully within bone hole 91. Shuttling links 41, 41a extend on each side of soft tissue 80.

FIG. 7: Tail 21 of repair suture 20 is loaded through loop 43 of the hybrid link 41.

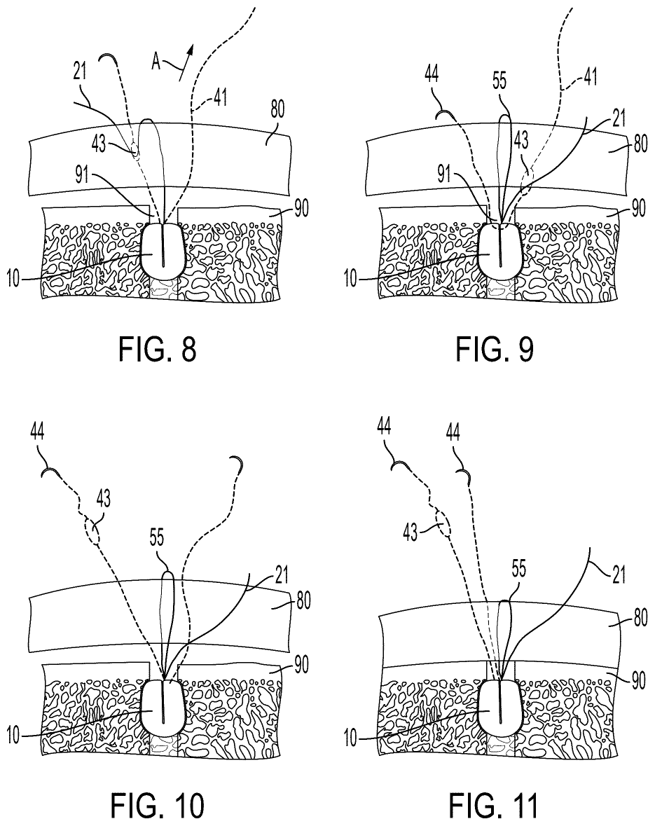

FIG. 8: Hybrid link 41 is pulled in the direction of arrow A to shuttle the repair suture 21, 20 down through the anchor 50 and through the sheath 10.

FIG. 9: The hybrid link 41 is only pulled enough to pass the repair suture 21, 20 through the anchor sheath 10. When the repair suture 21, 20 is passed through the anchor sheath it forms a splice and an adjustable, flexible, knotless, tensionable loop 55. FIG. 9 shows the link not completely pulled through.

FIG. 10: The hybrid link 41 and loop 43 can then be pulled back to even out the sutures for knot tying.

FIG. 11: The hybrid link sutures can be passed around or through the tissue 80. The suture end 21 of flexible coupler 20 can be tensioned and cut. Tensioning of suture end 21 and loop 55 approximates soft tissue 80 to tissue 90. In some implementations, suture leg 21 can be used for other tissue repairs that can include knotted repairs, knotless repairs, lateral tissue compression conducted with accompanying interference anchors, etc.

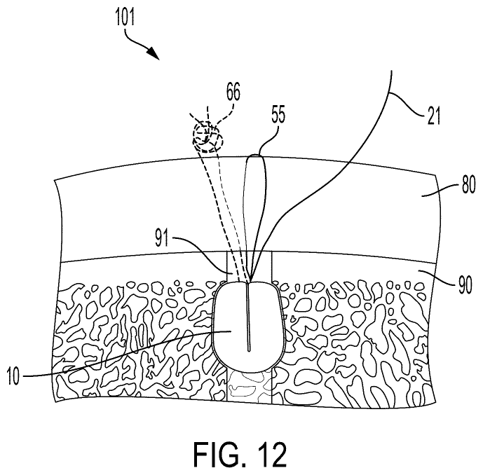

FIG. 12: The sliding hybrid link sutures can be tied in one or more knots 66 for final repair 101. Alternatively, the hybrid link sutures can be employed for additional surgical steps, for example, employed with additional surgical constructs. Additional surgical constructs may include soft suture anchors or hard-body anchors such as SwiveLock® anchors and/or PushLock® anchors, among others.

The knotless FiberTak® anchor with hybrid shuttling link constructs 100, 200, 300, 400 detailed above allow the shuttle link 40 to be repurposed into a repair suture instead of just being discarded after conversion of the repair suture 20, 21 and thrown. The disclosed link 40 with the loop/eyelet 43 in the mid-portion of the shuttling suture 41 can be engineered with either a bifurcated braid or a spliced eyelet similar to the FiberRing™ suture eyelet in the FiberRing™ suture construct. The new disclosed hybrid shuttling link 40 allows the surgeon to add an additional point of fixation to increase the strength of the repair construct. The hybrid link 40 allows for more advanced stitching configurations where tissue quality may be compromised. The hybrid link also could function as an InternalBrace™ suture that can span to another suture anchor.

The new disclosed hybrid link 40 is novel as the link is not wasted; it can become part of the repair construct after conversion of the repair suture. A variety of configurations are possible: single, double or multiple loops constructs; multiple constructs depending on the location of the loops (loop closer to the end of the suture v. toward the middle); constructs with one or more attaching needle(s) to the shuttling link tails; constructs with hybrid links made out of round suture, tape suture, bifurcated suture, or combinations thereof.

If more than one flexible coupler 20 and shuttling link 40 are employed, the plurality of flexible couplers and shuttling links can reside within the sheath 10 independent from each other or attached to (spliced to) each other. The flexible couplers may be similar to each other or different from each other. The shuttling links may be similar to each other or different from each other. In some implementations, the flexible coupler can be bifurcated or furcated from the sheath 10 to provide a plurality of knotless loops around tissue.

In some implementations, the flexible coupler 20 may come out of the cannulated sheath 10 and be braided to a smaller diameter round suture 21 that is passed back through the sheath. The braided suture may be furcated/branched/forked (for example, bifurcated or trifurcated, etc.) to provide a plurality of passing sutures.

In some implementations, end 21 of flexible coupler 20 may be a tapered end. In some implementations, the diameters of end 21 may be smaller than the diameters of flexible coupler 20. In some implementations, flexible coupler 20 may be a single strand suture. In some implementations, flexible coupler 20 may be a single braided suture.

The constructs, systems, and assemblies of the present disclosure may be employed in numerous knotless soft tissue repairs and fixations, for example, fixation of soft tissue to bone. In some implementations, a surgical construct 100, 200, 300, 400 may provide first tissue to second tissue fixation, for example, fixation of soft tissue 80 (ligament, tendon, graft, etc.) to bone 90 in a simple, fast and economical manner.

The fixation devices 100, 200, 300, 400 detailed above are soft anchors formed of “soft” materials, such as suture materials, yarns, fibers, filaments, strings, fibrils, strands, etc., or any combination of such materials. The soft materials may be synthetic or natural materials, or combinations of synthetic and natural materials, and may be bio-degradable or non-degradable, and may be elastic or non-elastic within the scope of this disclosure. These soft materials confer the ability to be inserted into bone sockets/holes/tunnels and bunch together, collapse, expand and/or change shape to fixate within the socket/hole/tunnel. In some implementations the soft anchor 100, 200, 300, 400 is made exclusively of soft, suture-based materials. Soft anchor 100, 200, 300, 400 can be an “all-suture” construct.

In some embodiments, the soft anchor 100, 200, 300, 400 includes a cannulated sheath and at least one flexible strand attached to the sheath. The flexible strand can form one or more adjustable, flexible, tensionable loops. At least one of the loops can be knotless. At least one of the loops can be knotted. At least one flexible strand can be slidable relative to the sheath. The slidable flexible strand can form at least one knotted loop around or through soft tissue. At least one flexible strand can be fixed relative to the sheath. The flexible fixed strand can form at least one knotless loop around or through soft tissue.

Soft anchor 100, 200, 300, 400 is configured for use in various soft tissue repairs or fixations and can be fixated inside bone or over bone for attaching tissue (e.g., ligament, tendon, graft, etc.) to bone. For example, the soft anchor 100, 200, 300, 400 can be used in conjunction with a variety of orthopedic surgical repairs, including but not limited to rotator cuff repairs, Achilles tendon repairs, patellar tendon repairs, ACL/PCL reconstructions, hip and shoulder reconstructions, among many others. The fixation can be on or over bone.

A soft suture anchor 100 comprises: (i) a cannulated sheath 10 having a length, a first end 11a, and a second end 11b; (ii) a flexible coupler 20 attached to the cannulated sheath 10, the flexible coupler 20 having a free end 21, the flexible coupler 20 being fixed relative to the sheath 10; and (iii) a hybrid link 40 slidable through the sheath 10 and configured to act both as a shuttling link and as a repair suture.

A soft suture anchor 100, 200, 300, 400 comprises: (i) a cannulated sheath 10 having a length, a first end 11a, and a second end 11b; (ii) a plurality of flexible couplers 20 attached to the cannulated sheath 10 and having a free end 21, the flexible couplers 20 being fixed relative to the sheath 10; and (iii) a plurality of hybrid links 40 slidable through the sheath 10 and configured to act both as a shuttling link and as a repair suture.

A method of tissue reconstruction 101 comprises: (i) securing a surgical construct 100 into tissue 90, wherein the construct 100 includes a soft anchor sheath 10 with a fixedly attached flexible coupler 20 and a slidably attached hybrid link 40; and (ii) forming at least two adjustable, tensionable loops around tissue 80 to be attached to the tissue 90 with the flexible coupler 20 and hybrid link 40. The method may further comprise employing the hybrid link 40 for additional surgical procedures. The additional surgical procedures may include passing the hybrid link 40 through or around tissue 80; forming at least one knot; and/or employing the hybrid link 40 with additional fixation devices. The additional fixation device can be a knotless anchor. The additional fixation device can be a hard body anchor. The additional fixation device can be a soft anchor. The flexible coupler 20 can consist essentially of elastic suture.

Tissue reconstruction can be any of small joint repairs, rotator cuff repairs, Achilles tendon repair, patellar tendon repair, ACL/PCL reconstruction, hip and shoulder reconstruction procedures, and applications for elastic suture used in or with suture anchors. Particular applications include repairs of the medial patellofemoral ligament (MPFL) repairs of the knee where the surgeon utilizes FiberTak® anchors and constructs 100, 200, 300, 400 to secure a suture tape (FiberTape® suture tape) that acts like a “brace” to support the repaired ligament, providing additional stability during the healing process; essentially augmenting the primary MPFL repair by increasing the area of tissue approximation around the repair site.

The surgical constructs and repair methods of the present disclosure can be employed in tissue repairs that may not involve knot tying, for example, for use with suture anchors (such as PushLock® and/or SwiveLock® suture anchors) or for knotless arthroscopic suture repairs (such as knotless single row rotator cuff repair, or SpeedBridge™ repairs using no knots and only suture passing steps), among many others.

Flexible coupler 20 and shuttling link 40 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided optionally with at least one other elastic fiber, natural or synthetic, to form lengths of suture material. Flexible coupler 20 and shuttling link 40 can also include suture tape such as FiberTape® suture tape (as disclosed in U.S. Pat. No. 7,892,256, the disclosure of which is incorporated in its entirety herewith) or collagen tape, or wide “tape like” material, or combinations thereof.

Flexible coupler 20 and shuttling link 40 can consist essentially of suture material and elastane, or combination of suture material and elastane and other materials such as long chain synthetic polymers like polyester and nylon, or materials such as PET, silk nylon or absorbable polymers, or coating materials (such as wax, silk, or silicone products), among many others. Flexible coupler 20 and shuttling link 40 can consist of strands with cross-sections of various forms and geometries, including round, oval, rectangular, or flat, among others, or combinations of such forms and geometries. In some implementations, at least one flexible coupler 20 and shuttling link 40 can be provided as a suture which is braided, knitted or woven. Flexible coupler 20 and shuttling link 40 can consist essentially of elastic suture.

Flexible coupler 20 and shuttling link 40 can be further attached to one or more fixation devices such as any anchors, for example, knotted anchors, knotless anchors, or all-suture anchors, or any devices that confer secure attachment and fixation of soft tissue over bone. The fixation device can be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

Parts or all of soft anchor 100, 200, 300, 400 including sheath 10, shuttling link 40 and flexible coupler 20 can be also coated and/or provided in different colors. In some implementations, parts (or all) of sheath 10, shuttling link 40 and flexible coupler 20 can be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the construct, pliability, handleability or abrasion resistance, for example.

Parts or all of sheath 10, shuttling link 40 and/or flexible coupler 20 can be also provided with tinted tracing strands, or otherwise contrast visually with other parts of the construct, which remain a plain, solid color, or displays a different tracing pattern, for example. Various structural elements of the surgical constructs may be visually coded, making identification and handling of the suture legs simpler. Easy identification of suture in situ is advantageous in surgical procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A soft anchor comprising:
a cannulated sheath having a length, a first end, and a second end;
a first flexible coupler fixedly attached to the cannulated sheath, the first flexible coupler having a free end; and
a second flexible coupler attached to the cannulated sheath and to the first flexible coupler, the second flexible coupler having two tails and being configured to slide through the cannulated sheath, the second flexible coupler including at least one closed loop at one of the two tails.

2. The soft anchor of claim 1, wherein the at least one closed loop is located at about a mid-length of the second flexible coupler.

3. The soft anchor of claim 1, wherein at least one of the two tails of the second flexible coupler is provided with a needle.

4. The soft anchor of claim 3, wherein each of the two tails of the second flexible coupler is provided with a needle and with a closed loop located between the needle and the cannulated sheath.

5. The soft anchor of claim 1, wherein the cannulated sheath is a tubular sheath secured into bone, wherein the free end of the first flexible coupler passes through the closed loop of the second flexible coupler and then through the tubular sheath at least once to form a splice and an adjustable, flexible, tensionable, knotless loop around soft tissue to be secured to the bone, and wherein the second flexible coupler forms a knotted loop around the soft tissue to be secured to the bone.

6. The soft anchor of claim 1, wherein the soft anchor is an all-suture soft anchor and the first flexible coupler is round suture.

7. The soft anchor of claim 6, wherein the second flexible coupler is a shuttling device that includes round suture and suture tape.

8. The soft anchor of claim 1, wherein the cannulated sheath is elastic.

9. The soft anchor of claim 1, wherein the soft anchor is manufactured as a one-piece construct.

10. The soft anchor of claim 1, wherein the first flexible coupler is furcated into a plurality of repair sutures.

11. A soft anchor repair system, comprising:
a flexible tubular sheath having a first end and a second end;
a first plurality of flexible couplers extending through at least a portion of the flexible tubular sheath and fixedly attached to the flexible tubular sheath; and
a second plurality of flexible couplers extending through at least a portion of the flexible tubular sheath and slidably attached to the flexible tubular sheath,
wherein the first plurality of flexible couplers form at least one adjustable, knotless loop around soft tissue to be attached to bone, and wherein the second plurality of flexible couplers form at least one knotted loop around the soft tissue to be attached to the bone.

12. The soft anchor repair system of claim 11, wherein the flexible tubular sheath is a suture tube.

13. The soft anchor repair system of claim 11, wherein each of the first plurality of flexible couplers passes through a closed eyelet of each of the second plurality of flexible couplers and then through the flexible tubular sheath to form the at least one adjustable, knotless loop.

14. The soft anchor repair system of claim 11, wherein the first plurality of flexible couplers extend through less than an entire length of the flexible tubular sheath.

15. A method of tissue repair, comprising:
securing a soft anchor sheath of a soft suture anchor within a first tissue, the soft anchor including a first flexible coupler fixedly attached to the soft anchor sheath, and a second flexible coupler attached to the soft anchor sheath and to the first flexible coupler, the second flexible coupler being configured to slide through the soft anchor sheath, the second flexible coupler including two opposing tails and a closed loop at one of the opposing tails;
forming, with the first flexible coupler, a knotless loop around second tissue to be attached to the first tissue; and
forming, with the second flexible coupler, a knotted loop around the second tissue to be attached to the first tissue.

16. The method of claim 15 wherein forming the knotted loop occurs after forming the knotless loop.

17. The method of claim 15, further comprising:
passing a free end of the first flexible coupler through the closed loop of the second flexible coupler; and
subsequently, pulling the second flexible coupler to slide through the soft anchor sheath and to pass the free end of the first flexible coupler through the soft anchor sheath to form a splice and the knotless loop.

18. The method of claim 15, wherein the tissue repair is a knee, foot, ankle, hand or wrist repair.

19. The method of claim 15, wherein the tissue repair is an InternalBrace repair.

20. The method of claim 15, wherein the tissue repair is rotator cuff repair.
